(19) United States
(12) Patent Application Publication
(10) Pub. No.: US 2013/0197415 A1
(43) Pub. Date: Aug. 1, 2013

(54) SYSTEM FOR SPINAL DECOMPRESSION

(71) Applicant: Gary D. Piaget, East Sound, WA (US)

(72) Inventor: Gary D. Piaget, East Sound, WA (US)

(21) Appl. No.: 13/363,756

(22) Filed: Feb. 1, 2012

(51) Int. Cl. A61F 5/00

(52) U.S. Cl. 602/36

(57) ABSTRACT

A spinal decompression system for the treatment of low back pain arising from lumbar disc disease that horizontally separates the vertebra by effectively applying the desired traction to relieve pain and stimulate healing by opening the pathway to the disc while maintaining the basic comfort of the patient. The system employs horizontal separation via a cam that is positioned between a stationary platform and a traction harness that moves relative to the platform. When the cam is activated it pushes the harness relative to the stationary platform. The cam action separates the vertebra by applying traction force to the harness relative to the table support section. The cam can be actuated using a lever arm, a hand cranked worm drive, hand wheel or an electric motor. To control the pressure being exerted on the spine, the cam drive is spring loaded to allow for the relief of any over pressure.

### BACKGROUND OF THE INVENTION

The present invention relates generally to a spinal decompression system wherein traction is applied to the spine. More specifically, the present invention relates to a spinal decompression system for the treatment of low back pain arising from lumbar disc disease that employs a table arrangement that horizontally separates the vertebra by effectively applying the desired traction to relieve pain and stimulate healing by opening the pathway to the disc while maintaining the basic comfort of the patient.

Traction therapy, also referred to as spinal decompression, is widely used to relieve pressure on inflamed or enlarged nerves and spinal discs. While traction is applicable to any part of the body, cervical and lumbar or spinal traction are the most common. It is known in the art that spinal decompression therapy has proven beneficial to most patients. When correctly performed, spinal traction can cause distraction or separation of the vertebral bodies, a combination of distraction and gliding of the facet joints, tensing of the ligamentous structures of the spinal segment, widening of the intervertebral foramen, straightening of spinal curvature and stretching of the spinal musculature. Depending on the injury and/or condition being treated, the traction component of physical therapy may require multiple sessions per week for a prolonged period of time.

To apply effective cervical traction, a traction force up to approximately 222 N (50 lbs.) must be applied. Lumbar traction typically requires application of a force equal to half of the patient's body weight, or about 333-667 N (75-150 lbs.). The problem is that the equipment necessary for performing such traction, however, has typically been expensive and thus only available to patients in a therapist's office. The typical machine used by chiropractors, for example, costs around $25,000. As a result, most traction systems commonly utilized have, for the most part, been relatively ineffective because patients cannot afford the treatments, or must travel to a doctor's office to receive the treatments. Therefore in most cases, bed rest is suggested as a means to healing, which in most cases provides only immediate relief, requiring extended periods of time in bed rather than actually applying the desired traction to provide real healing. In addition, the known devices have generally been large cumbersome, standalone units or traction units designed to attach to hospital beds and being both difficult to adjust for the attendant and uncomfortable for the patient.

Attempts to create a sufficiently low cost portable traction device for home use have thus far produced unsatisfactory results. A number of portable traction devices utilize pneumatic or hydraulic cylinders to create the traction force. Hydraulic cylinders have the disadvantage of the weight of the hydraulic fluid and the potential for leakage. Pneumatic cylinders with low pressure inputs typically cannot maintain an adequate traction force for a sufficient period of time to be effective in a traction device. In an attempt to overcome these deficiencies, some of these devices utilize an automatic pumping device triggered by a pressure sensing device to supply additional compressed air so that a constant level of traction force is maintained. These pump and sensor configurations add cost, weight and complexity to the traction device.

Other attempts have been made to provide low-cost, light weight portable, cervical traction or spinal decompression devices for home use by a patient. These devices employ air bags that inflate under the spine. The air input pumps used on some traction devices also exhibit a number of shortcomings. For example, bulb-type air pumps produce relatively small input pressures. A small female patient can generate only about 483 kPa (7 psi) of pressure using a bulb-type pump. Consequently, small input pressure devices require large diameter cylinders to generate the necessary output traction forces.

In some arrangements a carriage is slidable along a portion of a support structure parallel to a longitudinal axis. The carriage includes a restraining mechanism adapted to releasably restrain a portion of a patient's body to the carriage. The pneumatic traction force generating apparatus is a larger diameter cylinder, that when used with low pressure input devices, are more prone to leak, thereby further complicating the problem of maintaining a constant traction force for a prolonged period of time. The cylinder is operatively coupled to move the carriage relative to the support structure when in a pressurized state. The pneumatic traction force generating apparatus is adapted to maintain a generally static traction force during a treatment period when in the pressurized state without additional pressurized air being supplied. A hand pump operated by the patient is fluidly connected to the pneumatic traction force generating apparatus to inject pressurized air. The hand pump is capable of injecting at least 138 kPa (20 psi) of pressure into the pneumatic traction force generating apparatus. It should be noted that the recommended amount of minimum traction decompression pressure should be equal to one half of the patient's body weight. Therefore these existing traction devices do not supply even the minimum amount of decompression pressure required for adequate treatment. A pressure relief mechanism operated by the patient is adapted to release pressure from the pneumatic traction force generating apparatus. However, these methods are not as good as the horizontal separation employed in the very expensive machines.

Therefore, there is a need for a low cost, light weight portable traction device utilizing a pneumatic traction force generating apparatus that can maintain a traction force of an adequate magnitude for a prolonged period of time. There is a further need for a spinal decompression system for the treatment of low back pain arising from lumbar disc disease that horizontally separates the vertebra by effectively applying the desired traction to relieve pain and stimulate healing by opening the pathway to the disc while maintaining the basic comfort of the patient.

### BRIEF SUMMARY OF THE INVENTION

In this regard, the present invention provides for a spinal decompression system for the treatment of low back pain arising from lumbar disc disease that horizontally separates the vertebra by effectively applying the desired traction to relieve pain and stimulate healing by opening the pathway to the disc while maintaining the basic comfort of the patient.

In contrast to the prior art, the present concept employs horizontal separation by means of a cam that is positioned within a support platform that is stationary. The cam in turn displaces a body traction harness that is able to move. When the cam is activated it pushes against the harness relative to the stationary platform that is supporting the lower back. In this manner the cam action separates the vertebra by pushing the harness that is affixed to the user relative to the stationary table. It should be noted that the user attaches the harness at the hips and the upper body is held in place by any means known in the art such as arm-pit posts attached to the stationary platform or a chest harness arrangement.

The cam can be actuated in many ways, from a lever arm, to a hand cranked worm drive, to a hand wheel, to an electric motor geared to the cam or support shaft. To control the pressure being exerted on the spine, so there is no possibility of “over jacking” the spine a cam wheel that rides on and drives the cam would be spring loaded to allow the release of any over pressure.

It is therefore an object of the present invention to provide a low cost, light weight portable traction device utilizing a traction force generating apparatus that can maintain a traction force of an adequate magnitude for a prolonged period of time. It is a further object of the present invention to provide a spinal decompression system for the treatment of low back pain arising from lumbar disc disease that horizontally separates the vertebra by effectively applying the desired traction to relieve pain and stimulate healing by opening the pathway to the disc while maintaining the basic comfort of the patient.

These together with other objects of the invention, along with various features of novelty which characterize the invention, are pointed out with particularity in the claims annexed hereto and forming a part of this disclosure. For a better understanding of the invention, its operating advantages and the specific objects attained by its uses, reference should be made to the accompanying drawings and descriptive matter in which there is illustrated a preferred embodiment of the invention.

### BRIEF DESCRIPTION OF THE DRAWINGS

In the drawings which illustrate the best mode presently contemplated for carrying out the present invention:

FIG. 1 is a perspective view of a traction device of the present invention;

FIG. 2 is a partial cut-away plan view of the traction device of the present invention in the relaxed position;

FIG. 3 is a partial cut-away plan view of the traction device of the present invention in the tensioned position; and

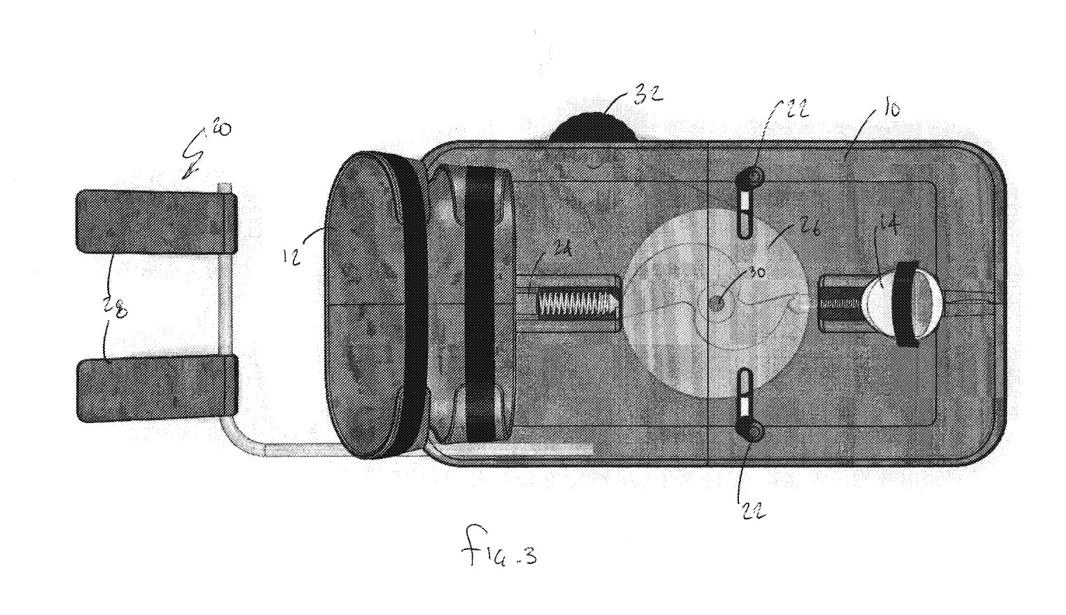

FIG. 4 is a perspective view of the cam tensioning system of the present invention.

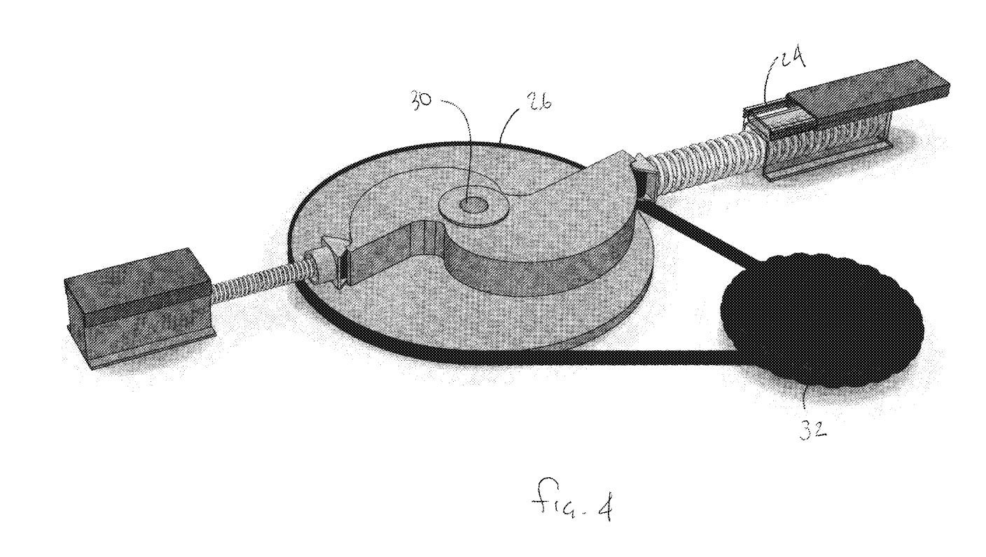

### DETAILED DESCRIPTION OF THE INVENTION

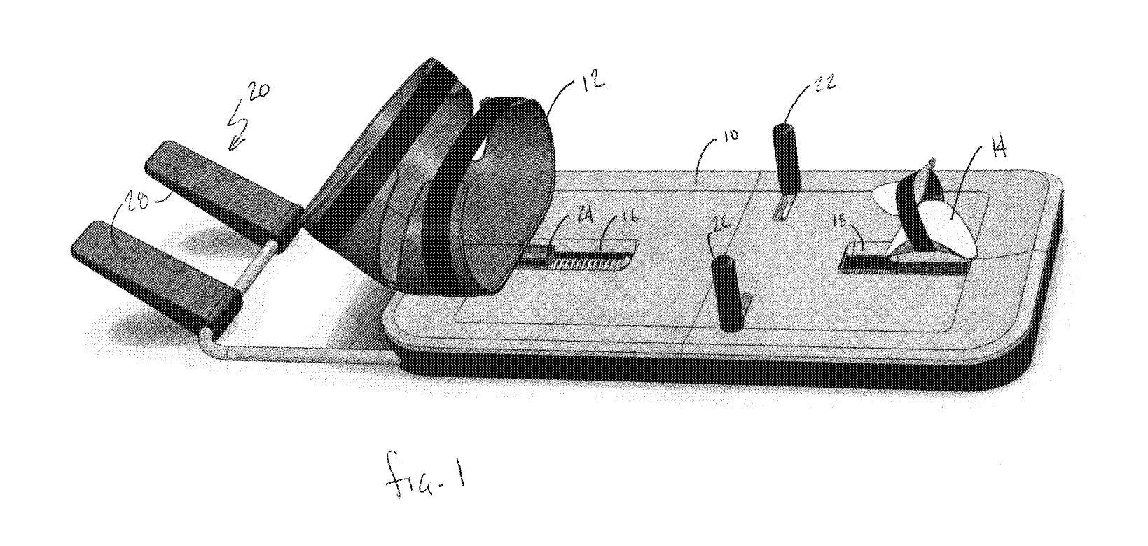

Now referring to the drawings, the spinal decompression system for the treatment of low back pain arising from lumbar disc disease that horizontally separates the vertebra by effectively applying the desired traction to relieve pain and stimulate healing by opening the pathway to the disc while maintaining the basic comfort of the patient is shown and generally illustrated in the figures. As can be seen at FIG. 1, the principal component of the system is a table that includes a body support platform 10 and at least one traction harness 12 that is able to move relative to the platform 10. In this manner, the present concept achieves horizontal separation of the harness by means of a cam that is positioned therebetween. When the cam is activated it pushes the harness away from a stationary platform that is supporting the user. In this manner the cam action separates the vertebra by pushing the harness 12 relative to the support table 10. Further a secondary traction harness 14 can be seen and is provided to apply neck traction pressure if needed or desired.

The foundation or base that supports the platform and the platform itself may be made by any appropriate method or from any known material in the art. Preferably the base and platform 10 are made from a polymer material employing a blow molding or injection molding process. Further, while the base and support platform may be made monolithically as a single component they may also be formed as two pieces that are joined to one another using any means known in the art. The platform 10 further houses a first track 16 along which the traction harness 12 is displaced, a second track 18 along which the secondary traction harness 18 is displaced and the cam assembly that applies the traction force as will be described in detail below.

In the preferred embodiment, the support table 10 is particularly configured and shaped ergonomically in a manner that supports the body of the user. Further support is achieved with the optional addition of a leg support arrangement 20. Although not required, preferably the support table 10 has an in mold contour or cavity that is suited to fit the body shape better. Still further the support table 10 is preferably long enough to support the body of the user from the head to mid-thigh although longer or shorter lengths may still be employed and still fall within the scope of the present invention.

The body of the user can be held stationary and in place on the support table in many ways known in the art. For example, as shown in FIG. 1, arm-pit posts 22 may be affixed to the upper surface of the support table 10 to engage under the user's arm-pits between their torso and arms. Further a chest harness may be used to provide a more comfortable restraint for the user. Still further, a chest harness that fits under the arms and over the chest having a singular large snap-engage buckle may be employed to restrain the upper portion of the user's body on the support table 10. The harness is shape-designed to fit the contour of the arm pits and chest and is filled with a material that self-contours for support and comfort.

To apply traction force to the user, a decompression harness 12 is employed in combination with the upper body supports 22. The decompression harness 12 is designed to contour-fit the user. The decompression harness 12 is imbedded in the support table in a molded track. The back area of the harness is fairly large to give good and proper support to the users spine, but to also allow for smooth movement over the support base 10. The harness 12 is preferably formed from a material such that the bottom surface of the decompression harness 12, where it contacts the upper surface of the support table 10 mitigates friction at this area. The top surface of the decompression harness lies at or above the surface level of the support table, so the harness provides a comfortable feel for the user at the lower spine area. The top surface of the support table is preferably mold contoured to fit the contour of the harness.

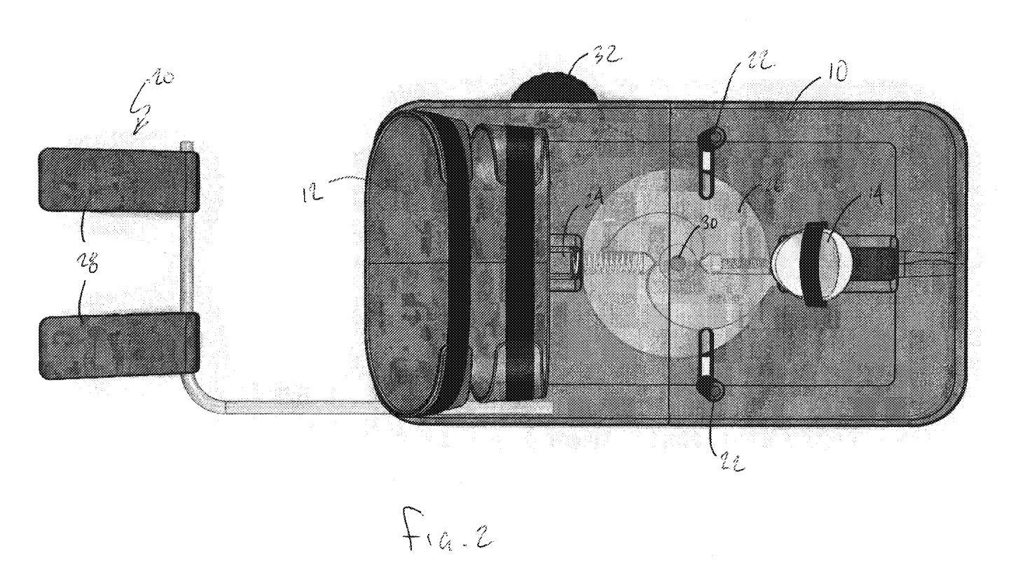

Turning to FIG. 2, a track 16 is formed to lie in a support trough beneath the decompression harness 12. An actuator 24 on the harness 12 rides in the track 16 which allows the harness 12 to be in contact with the cam 26 at all times. Further the harness/track is preferably spring loaded so the harness will return to the start-position as the cam pressure is removed. Further, to facilitate the various heights of the users the harness 12 can be adjusted upward or downward on the track 16 changing the distance between the harness 12 and the upper body support.

In connection with the harness system, leg and foot pads 28 may also be employed within the leg support 20 to locate and hold the legs in the proper position, knees bent upward. This position flattens the lower spine against the harness so the decompression-pull, separates the vertebra in a straight line.

The actuation cam 26 is located below the support deck 10 about the center line of the spine and is positioned at the location of the user's the lower back.

The cam 26 is supported in the table 10 by a support shaft 30 that allows the cam to rotate about the shaft. As the cam 26 rotates, it pushes the actuator 24 on the decompression harness 12 away, downward relative to the user's body, thereby applying traction force and decompressing the spine of the user. It is preferred that the actuator 24 is spring loaded and is located at the upper end of the harness track system. The lower hip harness 12 is attached to the track and the spring loaded actuator 24 will absorb any, “over pressure”, by moving away from the cam 26 while still in full contact with the cam. The spring could be, compressive adjustable, to allow for more finite pressure adjustment.

The cam can be actuated in many ways, from a lever arm, to a hand cranked worm drive, to a hand wheel, to an electric motor geared to the cam. The choice of drive will be determined by product cost and marketing, what is the best for the user, what is the price-point of the product. Historically there could be several different product options offered such as a hand operated system, or an automatic system, offering different price-points.

In an alternate embodiment the system of the present invention may include a two track system, which will provide much better support control and reduce side to side rocking of the harness. Similarly, a wider track system may be employed to impart the same benefit as compared to the two track system.

In another embodiment, the system will employ a duel-cam traction arrangement. Such an arrangement will facilitate the inclusion of a neck decompression harness 14 as well, to open the vertebra of the neck. Because the spine requires more energy to separate the vertebra than the neck, the cam 26 can be seen to employ two different pitches that are designed to apply only enough pressure to separate the vertebra safely. One of the cam 26 surfaces provides traction pressure for the spine and another surface having a different profile for the neck. The cam 26 may be singular and include two surfaces or could employ two cams stacked, one on top of the other, with a common support shaft. Each cam surface would have its own lobe design to facilitate the proper extension of both the neck and spine. The machine could be designed to allow for one or the other function, neck or spine, or both simultaneously. Still further, the cam(s) 26 can be removed and replaced by various size cams. This will make it possible to adjust the system for various heights in the user, as well as control the length of travel of the harness tracks, for both the neck and spine. Also one or two cams, stacked one on the other, can be used at the same time to allow for spine, or neck only, or both, spine and neck simultaneously. Finally, the cams may be mounted on a multi-sided support shaft that allows indexing of the cams relative to the shaft. This indexing provides for adjustment to increase the overall range of motion of each cam thereby allowing adjustment in the total tension the cams can exert.

It can therefore be seen that the present invention provides a low cost, light weight portable traction device utilizing a cam based traction force generating apparatus that can maintain a traction force of an adequate magnitude for a prolonged period of time. The present invention further provides a spinal decompression system for the treatment of low back pain arising from lumbar disc disease that horizontally separates the vertebra by effectively applying the desired traction to relieve pain and stimulate healing by opening the pathway to the disc while maintaining the basic comfort of the patient. For these reasons, the instant invention is believed to represent a significant advancement in the art, which has substantial commercial merit.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed:

1. A system for applying spinal traction, comprising:
a table for supporting a user;
a restraint system affixed to said table;
a cam system received within the table; and
a traction harness that is displaced away from said restraint system by actuation of said cam system.

2. The system of claim 1, wherein said table is molded from a polymer material.

3. The system of claim 1, wherein said table includes a recess in said upper surface that is ergonomically contoured to receive and support the body of a user.

4. The system of claim 1, wherein said restraint system is adjustable posts.

5. The system of claim 1, wherein said cam system further comprises:
at least one cam rotatably received on a support shaft; and
an actuator to rotate said cam.

6. The system of claim 5, wherein said traction harness is slidably engaged within a track in the top surface of the support table and is displaced by rotation of said cam.

7. The system of claim 5, wherein said actuator is selected from the group consisting of: a lever arm, hand cranked worm drive, hand wheels and an electric motor geared to the support shaft.

8. The system of claim 5, wherein said cam is removable and replaceable with a cam having a different profile.

9. The system of claim 5, wherein said cam is indexable relative to said support shaft.

10. The system of claim 1, wherein said cam system is spring biased to retract when not actuated.

11. The system of claim 1, further comprising:
a leg positioning assembly.

12. The system of claim 1, wherein said cam system further comprises:
a cam having two cam surfaces received on a support shaft within said platform; and
an actuator to rotate said cam.

13. The system of claim 12, wherein said actuator rotates said cam surfaces simultaneously.

14. The system of claim 12, wherein said actuator rotates said cam surfaces independently.

15. The system of claim 12, wherein one of the cam surfaces has a lobe shape to provide traction pressure for the spine and another cam surface has a lobe shape to provide traction pressure for the neck.

16. The system of claim 12, wherein said traction harness is slidably engaged within said track and displaced by rotation of said cam.

17. The system of claim 12, wherein said actuator is selected from the group consisting of: a lever arm, hand cranked worm drive and an electric motor geared to the support shaft.

18. The system of claim 12, wherein said cam is removable and replaceable with a cam having a different profile.
